(19) United States
(12) Patent Application Publication
(10) Pub. No.: US 2025/0275859 A1
(43) Pub. Date: Sep. 4, 2025

(54) LAYERED MEDICAL APPLIANCES AND METHODS

(71) Applicant: Merit Medical Systems, Inc., South Jordan, UT (US)

(72) Inventors: Wayne Mower, Bountiful, UT (US); Adam Wilhite, Herriman, UT (US); Darrell Skousen, Lehi, UT (US); Christopher Cindrich, Highland, UT (US); John Hall, Bountiful, UT (US); Yuma Medina, South Jordan, UT (US)

(21) Appl. No.: 19/067,173

(22) Filed: Feb. 28, 2025

Related U.S. Application Data
(60) Provisional application No. 63/560,428, filed on Mar. 1, 2024.

(51) Int. Cl. A61F 2/915; A61F 2/07; A61F 2/945

(52) U.S. Cl. CPC A61F 2/915; A61F 2/945; A61F 2002/072; A61F 2240/001

(57) ABSTRACT

Medical appliances may be formed of multilayered constructs. The layers of the constructs may be configured with various physical properties or characteristics. The disposition and arrangement of each layer may be configured to create an overall construct with a combination of the individual properties of the layers. Constructs may be used to create vascular prostheses or other medical devices.

### RELATED APPLICATIONS

This application claims priority to U.S. Provisional Patent Application No. 63/560,428, filed on Mar. 1, 2024 and titled, “Layered Medical Appliances and Methods,” which is hereby incorporated by reference in its entirety.

### TECHNICAL FIELD

The present disclosure relates generally to medical appliances, including medical appliances composed of two or more layers of material. Medical appliances within the scope of this disclosure may include porous layers, nonporous layers, fluid or cellular impermeable layers, and so forth. These layers may be included and/or arranged within the construct to affect the structural properties and/or biocompatibility of the medical appliance.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying figures in which:

FIG. 1 is a Scanning Electron Micrograph (SEM) (950×) of a serially deposited polytetrafluoroethylene (PTFE) fiber mat.

FIG. 2 is a Scanning Electron Micrograph (SEM) (950×) of an expanded polytetrafluoroethylene (ePTFE) mat.

FIG. 3A is a perspective cut-away view of a medical appliance.

FIG. 3B is a cross-sectional view of the medical appliance of FIG. 3A taken through line 3B-3B.

FIG. 3C is a cross-sectional view showing the layers of the medical appliance of FIG. 3A.

FIG. 3D illustrates a strip of ePTFE for helically wrapping around a medical appliance, according to one embodiment of the present disclosure.

FIG. 3E illustrates one or more strips of ePTFE helically wrapped around a medical appliance, according to one embodiment of the present disclosure.

FIG. 3F illustrates one or more strips of ePTFE helically wrapped around a medical appliance, according to one embodiment of the present disclosure.

FIG. 3G illustrates an exemplary stress strain curve of one or more strips of ePTFE, according to one embodiment of the present disclosure.

FIG. 4 is a cross-sectional schematic view of the medical appliance of FIG. 3A deployed within a body lumen.

FIG. 5 is a perspective view of a scaffolding structure for a stent-graft.

### DETAILED DESCRIPTION

Medical appliances may be deployed in various body lumens for a variety of purposes. Stents and/or stent-grafts may be deployed, for example, in the vascular system for a variety of therapeutic purposes, including the treatment of occlusions within the lumens of that system. The current disclosure may be applicable to stents, stent-grafts, or other medical appliances designed for the central venous (“CV”) system, peripheral vascular (“PV”) stents, abdominal aortic aneurysm (“AAA”) stents, bronchial stents, esophageal stents, biliary stents, coronary stents, gastrointestinal stents, neuro stents, thoracic aortic endographs, or any other stent or stent-graft. Further, the present disclosure may be equally applicable to other prostheses such as grafts, shunts, and so forth. Additionally, medical appliances comprising a continuous lumen wherein a portion of the longitudinal length is reinforced, for example by a metal scaffold, and a portion of the longitudinal length has no scaffold are also within the scope of this disclosure. Any medical appliance composed of materials herein described may be configured for use or implantation within various areas of the body, including vascular, cranial, thoracic, pulmonary, esophageal, abdominal, or ocular application. Examples of medical appliances within the scope of this disclosure include, but are not limited to, stents, vascular grafts, stent-grafts, cardiovascular patches, reconstructive tissue patches, hernia patches, general surgical patches, heart valves, sutures, dental reconstructive tissues, medical device coverings and coatings, gastrointestinal devices, blood filters, artificial organs, ocular implants, and pulmonary devices, including pulmonary stents. For convenience, many of the specific examples included below reference stent-grafts. Notwithstanding any of the particular medical appliances referenced in the examples or disclosure below, the disclosure and examples may apply analogously to any prostheses or other medical appliance.

As used herein, the terms stent and stent-graft refer to medical appliances configured for use within bodily structures, such as within body lumens. A stent or stent-graft may comprise a scaffolding or support structure, such as a frame, and/or a covering.

It will be readily understood that the components of the embodiments as generally described and illustrated in the Figures herein could be arranged and designed in a wide variety of different configurations. Thus, the following more detailed description of various embodiments, as represented in the Figures, is not intended to limit the scope of the disclosure, but is merely representative of various embodiments. While the various aspects of the embodiments are presented in drawings, the drawings are not necessarily drawn to scale unless specifically indicated.

The phrases” “coupled to” and “in communication with” refer to any form of interaction between two or more entities, including mechanical, fluid, and thermal interaction. Two components may be coupled to each other even though they are not in direct contact with each other. For example, two components may be coupled to each other through an intermediate component.

The directional terms “proximal” and “distal” are used herein to refer to opposite locations on a stent or another medical appliance. The proximal end of an appliance is defined as the end closest to the practitioner when the appliance is disposed within a deployment device which is being used by the practitioner. The distal end is the end opposite the proximal end, along the longitudinal direction of the appliance, or the end furthest from the practitioner. It is understood that, as used in the art, these terms may have different meanings once the appliance is deployed (i.e., the “proximal” end may refer to the end closest to the head or heart of the patient depending on application). For consistency, as used herein, the ends labeled “proximal” and “distal” remain the same regardless of whether the appliance is deployed.

The term “nominal diameter” is used herein to refer to a natural diameter of a medical appliance or expandable balloons. The nominal diameter of a medical appliance may be a manufacturing diameter of the medical appliance or a set diameter of the medical appliance fabricated from a shape memory material. The nominal diameter of an expandable balloon is an inflated diameter of the balloon when the balloon is expanded without deformation of the balloon.

The term “use diameter” is used herein to refer to a diameter of a medical appliance that is greater than the nominal diameter of the medical appliance. The use diameter provides a greater range of dilation than the simply the nominal diameter of the medical appliance. In some circumstances, the use diameter provides the medical practitioner options for treating a patient due to the fact that the use diameter of the medical appliance provides a greater range of dilation than the nominal diameter of the medical appliance.

The longitudinal direction of a stent or stent-graft is the direction along the axis of a generally tubular stent or stent-graft. In embodiments where an appliance is composed of a metal wire structure coupled to one or more layers of a film or sheet-like component, such as a polymer layer, the metal structure is referred to as the “scaffolding” or “frame,” and the polymer layer as the “covering” or “coating.” The terms “covering” and “coating” may refer to a single layer of polymer, multiple layers of the same polymer, or layers comprising distinct polymers used in combination. Furthermore, as used herein, the terms “covering” and “coating” refer only to a layer or layers which are coupled to a portion of the scaffold; neither term requires that the entire scaffold be “covered” or “coated.” In other words, medical appliances wherein a portion of the scaffold may be covered and a portion may remain bare are within the scope of this disclosure. Finally, any disclosure recited in connection with coverings or coatings may analogously be applied to medical devices comprising one or more “covering” layers with no associated frame or other structure. For example, a hernia patch comprising any of the materials described herein as “coatings” or “coverings” is within the scope of this disclosure regardless of whether the patch further comprises a frame or other structure. Similarly, a tubular graft or shunt may be comprised of the covering or layered materials recited herein, with no associated scaffolding structure.

Medical device coverings may comprise multilayered constructs, comprised of two or more layers which may be serially applied. Further, multilayered constructs may comprise nonhomogeneous layers, meaning adjacent layers have differing properties. Thus, as used herein, each layer of a multilayered construct may comprise a distinct layer, either due to the distinct application of the layers or due to differing properties between layers. As layers may be identified by their position, structure, or function, an individual layer may not necessarily comprise only a single material or a single microstructure.

Additionally, as used herein, “tissue ingrowth” and “cellular penetration” refer to any presence or penetration of a biological or bodily material into a component of a medical appliance. For example, the presence of body tissues (e.g., collagen, cells, and so on) within an opening or pore of a layer or component of a medical appliance comprises tissue ingrowth into that component, for example within openings or perforations along the body the medical appliance, not necessarily the proximal or distal openings of the medical appliance. Further, as used herein, “attachment” of tissue to a component of a medical appliance refers to any bonding or adherence of a tissue to the appliance, including indirect bonds. For example, tissue of some kind (e.g., collagen) may become attached to a stent covering (including attachment via tissue ingrowth), and another layer of biologic material (such as endothelial cells) may, in turn, adhere to the first tissue. In such instances, the second biologic material (endothelial cells in the example) and the tissue (collagen in the example) are “attached” to the stent covering.

In some instances, layers with porosities greater than six or eight micron, when combined with additional layers to create a more tortuous transmural path, may be impermeable to transmural migration of red blood cells across the combined layers, even if no single layer has a porosity less than eight micron.

Still further, composite constructs comprising various layers of various porosities may be impermeable to transmural migration of red blood cells. In some embodiments, a construct comprised of layers of varying porosities, coupled to a substantially nonporous layer may be impermeable to transmural migration of red blood cells.

Moreover, constructs within the scope of this disclosure may be cell impermeable to any cell type, meaning that less than 0.1% of cells which contact the construct (regardless of the cell type), will migrate across the construct wall. Similarly, constructs within the scope of this disclosure may be tissue impermeable, meaning that less than 0.1% of the mass or volume of tissue which contacts the construct will migrate across the construct wall.

In one example, a cell impermeable tubular multilayered construct was implanted in an animal host for 30 days. Upon removal of the multilayered construct, no measurable amount of tissue was present on the luminal surface of the construct. Accordingly, materials implanted in an animal host for 30 days with no measurable transmural cell or tissue migration are cellular and tissue impermeable, as those terms are used herein. It was also observed that standard ePTFE stent-graft exhibited tissue growth on the luminal surface when similarly implanted.

Additionally, materials or constructs may be impermeable to fluidic passage across the material wall. Materials or layers with pore sizes smaller than 0.5 micron are herein referenced as being impermeable to fluidic passage, or fluid impermeable across the material or layer.

The pore sizes associated with any of the embodiments above may refer to average pore size as further defined below. It may also refer to pore sizes determined by direct measurement techniques. The terms “generally” and “substantially” as used herein indicate that a parameter is within 5% of a reference parameter. Thus, two quantities termed as generally equivalent are within 5% of each other. Further, a generally or substantially impermeable membrane only varies from the defined pore sizes above by 5%.

Lumens within the circulatory system are generally lined with a single layer (monolayer) of endothelial cells. This lining of endothelial cells makes up the endothelium. The endothelium acts as an interface between blood flowing through the lumens of the circulatory system and the inner walls of the lumens. The endothelium, among other functions, reduces or prevents turbulent blood flow within the lumen. The endothelium plays a role in many aspects of vascular biology, including atherosclerosis, creating a selective barrier around the lumen, blood clotting, inflammation, angiogenesis, vasoconstriction, and vasodilation.

A therapeutic medical appliance which includes a covering of porous or semi-porous material may permit the formation of an endothelial layer onto the porous surface of the blood contact side of the medical device. Formation of an endothelial layer on a surface, or endothelialization, may increase the biocompatibility of an implanted device. For example, a stent which permits the formation of the endothelium on the inside diameter (blood contacting surface) of the stent may further promote healing at the therapeutic region and/or have longer-term viability. For example, a stent coated with endothelial cells may be more consistent with the surrounding body lumens, thereby resulting in less turbulent blood flow or a decreased risk of thrombosis, or the formation of blood clots. A stent or stent-graft which permits the formation of an endothelial layer on the inside surface of the stent may therefore be particularly biocompatible, resulting in less trauma at the point of application, fewer side effects, and/or longer-term device viability. Medical appliances including a covering of porous or semi-porous material may be configured to inhibit or reduce inflammatory responses by the body toward the tissue- contacting side of the medical appliance, for example. Mechanisms such as an inflammatory response by the body toward the medical appliance may stimulate, aggravate, or encourage negative outcomes, such as neointimal hyperplasia. For example, a device configured to permit tissue ingrowth and/or the growth or attachment of endothelial cells onto the blood-contacting side of the device may reduce the likelihood of negative flow characteristics and blood clotting. Similarly, a device so configured may mitigate the body's inflammatory response toward the material on, for example, the tissue-contacting side of the device. By modulating the evoked inflammatory response, negative outcomes such as the presence of bioactive inflammatory macrophages and foreign body giant cells may be reduced. This may aid in minimizing the chemical chain of responses that may encourage fibrous capsule formation surrounding the device and events stimulating neointimal hyperplasia.

Serially deposited fibers, such as rotational spun or electrospun materials, such as those described herein, may be used to comprise portions of medical appliances, such as stents, patches, grafts, and so forth. The present disclosure is applicable to any implantable medical appliance, notwithstanding any specific examples included below. In other words, though particular medical appliances, such as stents or patches, may be referenced in the disclosure and examples below, the disclosure is also analogously applicable to other medical appliances, such as those which comprise a covering or layer of polymeric material.

In some embodiments, serially deposited nanofibers (and/or microfibers) may be configured to permit interaction with nano-scale (and/or micro-scale) body structures, such as endothelial cells, red blood cells, collagen, and so forth.

Medical appliances may comprise two or more layers or materials. These layers, alone or in combination, may be designed or configured to impart various properties to the overall construct. For example, one or more layers, and/or the combined characteristics of two or more layers, may control the structural properties of the overall construct, such as tensile strength, burst strength, flexibility, hoop strength, resistance to radial compression, and so forth. Similarly, one or more layers, and/or the combined characteristics of two or more layers, may control the biocompatibility of the medical appliance. For example, porosity, fluid permeability, cellular permeability, and so forth may all affect the biologic response to a medical appliance deployed within a patient's body.

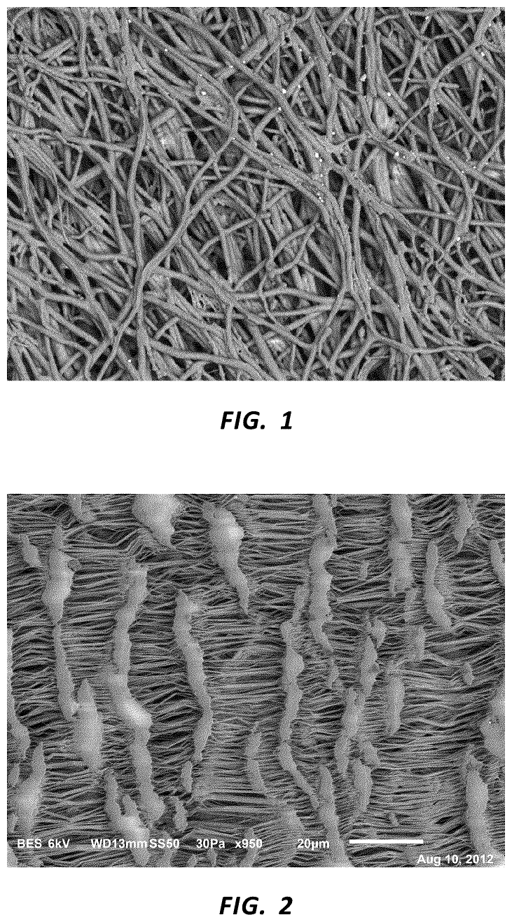

Various structures, including medical appliances and related components, may comprise serially deposited fibers. Serially deposited fibers may comprise polymeric fibers, ceramic fibers, and/or other materials. In some embodiments, soft or fluidic materials are deposited in elongate strands or fibers on a collector or substrate. After these fibers are deposited, the shape or structure of the mat or lattice of fibers may be set by, for example, hardening of the material of the fibers. For example, polymeric materials may be deposited as fibers in the form of a polymeric dispersion and then heated to remove the solvent component of the dispersion and to set the structure of the polymeric fibers. Similarly, polymeric materials may be serially deposited as fibers while the material is in a heated or molten state. Cooling of the collected fibers may tend to set the structure of the mat or lattice of fibers. The fibers comprising these mats or lattices may generally be on a micro scale (fibers which are between one micron and one millimeter in diameter) and/or generally on a nano scale (fibers which are smaller than one micron in diameter). FIG. 1 is an SEM (950×) of an exemplary serially deposited fiber mat. The fibers of the mat of FIG. 1 were deposited by rotational spinning of polytetrafluoroethylene (PTFE).

Serially deposited fiber mats or lattices refer to structures composed at least partially of fibers successively deposited on a collector, on a substrate, on a base material, and/or on previously deposited fibers. In some instances, the fibers may be randomly disposed, while in other embodiments the alignment or orientation of the fibers may be somewhat controlled or follow a general trend or pattern. Regardless of any pattern or degree of fiber alignment, because the fibers are deposited on the collector, substrate, base material, and/or previously deposited fibers, the fibers are not woven, but rather serially deposited. Because such fibers are configured to create a variety of structures, as used herein, the terms “mat” and “lattice” are intended to be broadly construed as referring to any such structure, including tubes, spheres, sheets, and so on. Furthermore, the term “membrane” as used herein refers to any structure comprising serially deposited fibers having a thickness which is smaller than at least one other dimension of the membrane. Examples of membranes include, but are not limited to, serially deposited fiber mats or lattices forming sheets, strips, tubes, spheres, covers, layers, and so forth.

Rotational spinning is one example of how a material may be serially deposited as fibers. One embodiment of a rotational spinning process comprises loading a polymer solution or dispersion into a cup or spinneret configured with orifices on the outside circumference of the spinneret. The spinneret is then rotated, causing (through a combination of centrifugal and hydrostatic forces, for example) the flowable material within the spinneret to be expelled from the orifices. The material may then form a “jet” or “stream” extending from the orifice, with drag forces tending to cause the stream of material to elongate into a small diameter fiber. The fibers may then be deposited on a collection apparatus, a substrate, or other fibers. Once collected, the fibers may be dried, cooled, sintered, or otherwise processed to set the structure or otherwise harden the fiber mat. For example, polymeric fibers rotational spun from a dispersion may be sintered to remove solvents, fiberizing agents, or other materials as well as to set the structure of the mat. In one embodiment, for instance, an aqueous polytetrafluoroethylene (PTFE) dispersion may be mixed with polyethylene oxide (PEO) (as a fiberizing agent) and water (as a solvent for the PEO), and the mixture rotational spun. Sintering by heating the collected fibers may set the PTFE structure, evaporate off the water, and sublimate the PEO. Exemplary methods and systems for rotational spinning can be found in U.S. patent application Ser. No. 13/742,025, filed on Jan. 15, 2013, and titled “Rotational Spun Material Covered Medical Appliances and Methods of Manufacture,” which is herein incorporated by reference in its entirety.

Rotational spinning processes and electrospinning processes may produce serially deposited fiber mats with differing characteristics. For example, as compared to electrospinning processes, rotational spinning may exhibit superior yield, performance, and scaling. Rotational spinning processes may exhibit greater repeatability, reliability, and quality compared to electrospinning. In other words, rotational spun fiber mats may exhibit more consistency within closer tolerances as compared to electrospun fiber mats. Rotational spinning may be more directly scalable to large operations. Whereas electrospinning on a large scale may entail high voltage and other difficulties that further introduce time, cost, and variability, rotational spinning may more directly and simply scale, producing more consistent fiber mats.

Electrospinning is another embodiment of how a material may be serially deposited as fibers. One embodiment of an electrospinning process comprises loading a polymer solution or dispersion into a syringe coupled to a syringe pump. The material is forced out of the syringe by the pump in the presence of an electric field. The material forced from the syringe may elongate into fibers that are then deposited on a grounded collection apparatus, such as a collector or substrate. The system may be configured such that the material forced from the syringe is electrostatically charged, and thus attracted to the grounded collection apparatus. As with rotational spinning, once collected, the fibers may be dried, cooled, sintered, or otherwise processed to set the structure or otherwise harden the fiber mat. For example, polymeric fibers electrospun from a dispersion may be sintered to remove solvents, fiberizing agents, or other materials as well as to set the structure of the mat. As in rotational spinning, one embodiment of electrospinning comprises electrospinning an aqueous PTFE dispersion mixed with PEO and water (as a solvent for the PEO). Sintering by heating the collected fibers may set the PTFE structure, evaporate off the water, and sublimate the PEO. Exemplary methods and systems for electrospinning medical devices can be found in U.S. patents application Ser. Nos. 13/826,618 and 13/827,790, both filed on Mar. 13, 2014, and both titled “Electrospun Material Covered Medical Appliances and Methods of Manufacture,” and U.S. patent application Ser. No. 13/360,444, filed on Jan. 27, 2012, and titled “Electrospun PTFE Coated Stent and Method of Use,” each of which is hereby incorporated by reference in its entireties.

Rotational spinning and/or electrospinning may be utilized to create a variety of materials or structures comprising serially deposited fibers. The microstructure or nanostructure of such materials, as well as the porosity, permeability, material composition, rigidity, fiber alignment, and so forth, may be controlled or configured to promote biocompatibility or influence interactions between the material and cells or other biologic material. A variety of materials may be serially deposited through processes such as rotational spinning and electrospinning: for example, polymers, ceramics, metals, materials which may be melt-processed, or any other material having a soft or liquid form. A variety of materials may be serially deposited through rotational spinning or electrospinning while the material is in a solution, dispersion, molten or semi-molten form, and so forth. The present disclosure may be applicable to any material discussed herein being serially deposited as fibers onto any substrate or in any geometry discussed herein. Thus, examples of particular materials or structures given herein may be analogously applied to other materials and/or structures.

Rotational spinning, electrospinning, or other analogous processes may be used to create serially deposited fiber mats as disclosed herein. Throughout this disclosure, examples may be given of serially deposited fiber mats generally, or the examples may specify the process (such as rotational spinning or electrospinning) utilized to create the serially deposited fiber mat. It is within the scope of this disclosure to analogously apply any process for creating serially deposited fibers to any disclosure or example below, regardless of whether the disclosure specifically indicates a particular mat was formed according to a particular process.

Expanded polytetrafluoroethylene (ePTFE) may also be used as a component of a layered medical appliance in some embodiments. ePTFE may be formed when a sheet of PTFE is heated and stretched. The sheet of ePTFE may be formed, for example, by extrusion or other methods. Heating and stretching of the PTFE sheet to form ePTFE changes the microstructure of the sheet, making it more porous and creating nodes of material with fibrils of material extending there between. U.S. Pat. No. 3,664,915 of W. L. Gore describes various processes for heating and stretching PTFE to create ePTFE. In some processes, the ePTFE will be expanded to a greater extent along a longitudinal direction as compared to a transverse direction. Thus some ePTFE mats may be described as having an axis of expansion, or the direction in which the majority of the expansion was done. In some instances the ratio of expansion in the longitudinal direction to expansion in a transverse direction may be between 10:1 and 20:1. FIG. 2 is an exemplary SEM (950×) of an ePTFE membrane.

In some applications, ePTFE may also be further processed after it has been initially formed. Some such processes may densify the ePTFE, reducing the porosity and increasing the strength. In some instances such post-processing may be used to contract fibrils extending between the nodes of the ePTFE to create a layer that is substantially impermeable to tissue and/or fluid. Thus, ePTFE layers may be configured with various permeability characteristics, including layers which are impermeable to cellular or tissue migration across the layer. Further, such post-processing may increase the strength of the layer due to the resulting work processing stresses created in the material.

Characteristics of mats, including serially deposited fibers and/or ePTFE, may be determined in a variety of ways. For example, internodal distance, or IND, of ePTFE can be used to characterize the degree of expansion and/or the porosity of the ePTFE. The internodal distance refers to the average distance between adjacent nodes of the membrane.

Percent porosity is another measurement that may be used to characterize membranes with porous sections. This method can be used, for example, to characterize ePTFE and/or serially deposited fibers. Percent porosity refers to the percent of open space to closed space (or space filled by fibers) in a membrane or mat. Thus, the more open the structure is, the higher the percent porosity measurement. In some instances, percent porosity may be determined by first obtaining an image, such as an SEM, of a material. The image may then be converted to a “binary image,” or an image showing only black and white portions, for example. The binary image may then be analyzed and the percent porosity determined by comparing the relative numbers of each type of binary pixel. For example, an image may be converted to a black and white image wherein black portions represent gaps or holes in the membrane while white portions represent the fibers or other structure of the membrane. Percent porosity may then be determined by dividing the number of black pixels by the number of total pixels in the image. In some instances, a code or script may be configured to make these analyses and calculations.

In some embodiments the “average pore size” of an ePTFE of serially deposited mat may be used as an alternate or additional measurement of the properties of the mat. The complex and random microstructure of serially deposited mats, for example, presents a challenge to the direct measurement of the average pore size of the mat. Average pore size can be indirectly determined by measuring the permeability of the mat to fluids using known testing techniques and instruments. Once the permeability is determined, that measurement may be used to determine an “effective” pore size of the serially deposited mat. As used herein, the “pore size” of a serially deposited mat and/or an expanded membrane refers to the pore size of a known membrane which corresponds to the permeability of the serially deposited or expanded fabric when measured using ISO standard 7198 for the permeability measurement. This standard is described in ISO 7198 “Water Entry Pressure” which is incorporated herein by reference. In some instances a water entry pressure test can be used as a quality control after configuring a mat based on other measurements such as percent porosity.

Further, average pore diameter and average pore area of an ePTFE or serially deposited mat may be calculated programmatically using software to analyze an image, such as an SEM. For example, an SEM may be evaluated using software analysis to measure various characteristics of a material layer. As part of this exemplary process, the SEM image may first be converted to a “binary image,” or an image showing only black and white portions, as discussed above. The binary image may then be analyzed, the fibers or other features of the layer identified, and characteristics determined by comparing the relative numbers and placements of each type of binary pixel. For example, an image may be converted to a black and white image wherein black portions represent gaps or holes in a serially deposited fiber mat while white portions represent the fibers of the mat. The software thus identifies the presence and position of fibers and pores or open portions of the fiber mat.

Characteristics such as fiber width and pore size may be determined by analyzing these binary images. Still further, characteristics of relative fibers, such as the number of fiber branches, intersections, bundles, fiber density, and so forth, may be determined through similar analysis. In some instances, a code or script may be configured to make various analyses and calculations. U.S. patent application Ser. No. 14/207,344, titled “Serially Deposited Fiber Materials and Associated Devices and Methods,” filed on Mar. 12, 2014, which is hereby incorporated by reference in its entirety, discusses various methods of characterizing and evaluating construct layers.

In determining average pore size, an image may be evaluated to distinguish between areas comprising fibers and open areas, such as by creating a binary image as discussed above. Pores, or areas within a fiber mat encapsulated by intersecting or branched fibers, may then be identified. To determine the average pore diameter, a large sample of pores may be randomly selected from the target image. In some instances, between 50 and 300 pores may comprise the sample. The diameter of a particular pore may be calculated by tracing multiple diameters of equal angular spacing around the pore through the centroid of the pore. In some embodiments, 30 such diameters were used to determine a calculated pore size. The measured diameters are then averaged to determine the calculated effective diameter of the pore. The area of each identified pore may also be computed based on the pixel area of each pore. Each pore identified for sampling may be manually checked to confirm proper identification of pores. The average pore diameter of a fiber mat may then be computed by averaging the calculated effective diameters of the identified pore. Again, material total porosity may also be determined by the percentage of dark pixels to light pixels in the image.

In many circumstances, a medical practitioner may implant a medical appliance in a patient's vasculature. The medical practitioner may expand the medical appliance to a nominal diameter to treat the patient's vasculature. In some instances, the medical practitioner may decide to expand the medical appliance beyond the nominal diameter of the medical appliance to a use diameter to treat the patient's vasculature. As used herein the “nominal diameter” refers to the designed or labeled diameter of a medical device. The “use diameter,” as used herein, is a diameter in which the medical appliance may be used beyond the nominal diameter of the medical appliance while maintaining the impermeability to tissue ingrowth of a medical appliance. In some embodiments, the use diameter may be less than the nominal diameter of the medical appliance. The medical practitioner may do this for a number of reasons. For example, the medical practitioner may decide that the nominal diameter of the medical appliance may not be sufficient to treat the vasculature and may expand the medical appliance beyond its nominal diameter up to the use diameter to better treat the vasculature. In addition, the use diameter provides a greater range of dilation for the medical practitioner to use without having to swap out medical appliances if the nominal diameter of the medical appliance is insufficient to the treat the patient's vasculature. In some embodiments, the use diameter is up to 40 percent greater than the nominal diameter.

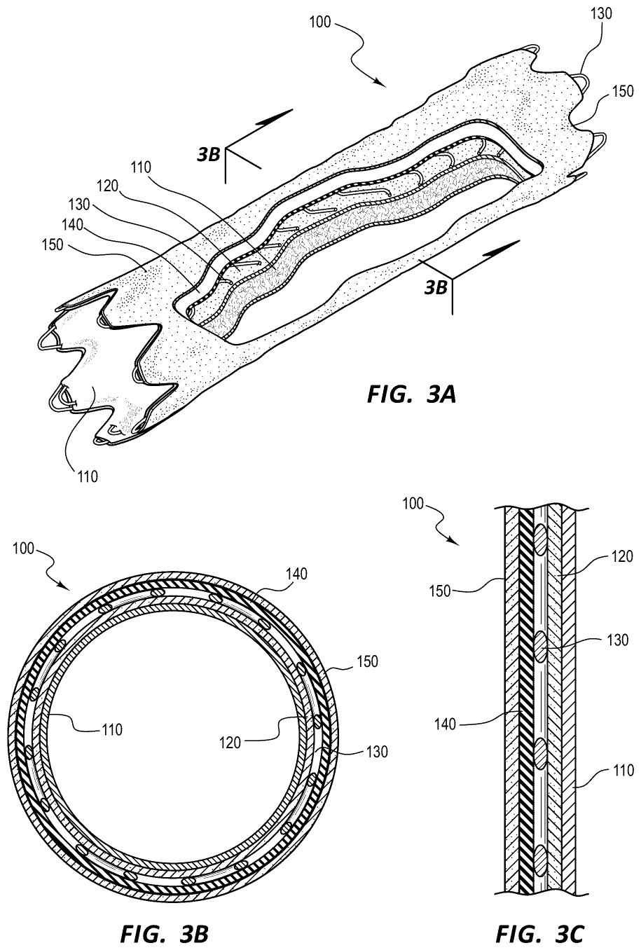

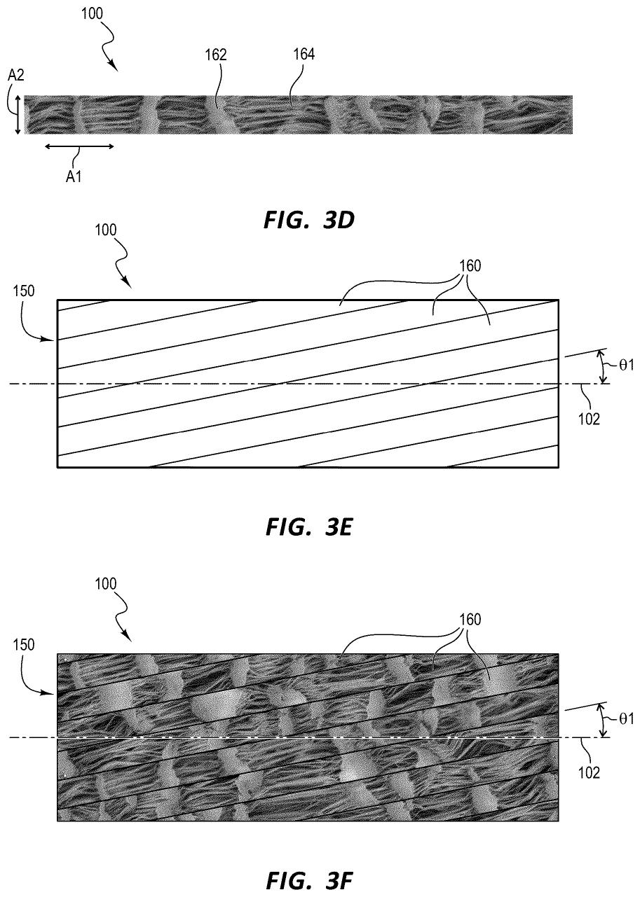

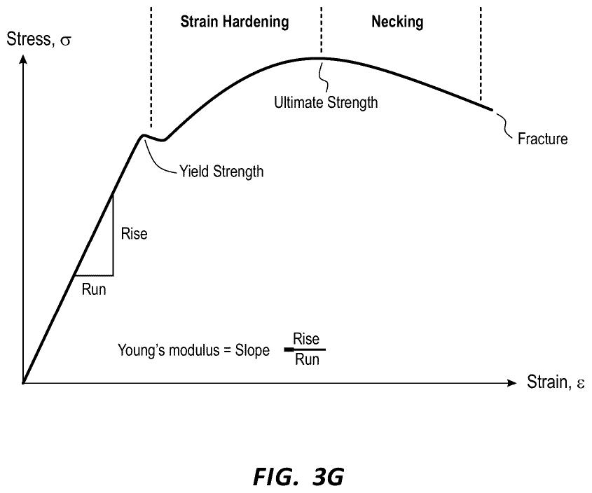

FIGS. 3A-3C are schematic depictions of a medical appliance 100. Specifically, FIG. 3A is a perspective cut-away view of the medical appliance 100. FIG. 3B is a cross-sectional view of the medical appliance 100 taken through line 3B-3B of FIG. 3A. FIG. 3C is another cross-sectional view showing the layers of the medical appliance 100.

The medical appliance 100 of FIGS. 3A-3C may be configured as a vascular stent-graft or a covered stent. In the illustrated embodiment, the medical appliance 100 is shown with four distinct layers 110, 120, 140, 150 disposed about a scaffolding structure 130. In the illustrated embodiment, the medical appliance 100 comprises a first layer 110 of rotationally spun PTFE. The rotationally spun PTFE first layer 110 may be an innermost layer of the medical appliance 100 and may define the luminal surface of the medical appliance 100. This luminal first layer 110 may be designed to interact with blood flow within the vasculature. For example, the microporous structure of the rotationally spun first layer 110 may be configured to accommodate or allow endothelial cell growth on the luminal surface of the medical appliance 100 when disposed within the vasculature of a patient. Alternatively or additionally, it is within the scope of this disclosure to use any serially deposited material on the luminal surface of the medical appliance 100. For example, rotational spun PTFE, electrospun PTFE, or other polymers may be used on this layer.

Serially deposited layers within the scope of this disclosure may comprise a wide variety of characteristics. For example, serially deposited layers with a percent porosity between 35% and 75%, including between 40% and 60%, and between 45% and 55%, are within the scope of this disclosure. Similarly, serially deposited layers with an average fiber diameter between 0.25 micron and 2.5 micron, including between 0.5 micron and 1.75 micron, and between 0.75 micron and 1.25 micron, are within the scope of this disclosure. Average pore diameter for serially deposited layers within the scope of this disclosure may range from one micron to five micron, including from two micron to four micron and from two micron to three micron. The average pore area of serially deposited layers within the scope of this disclosure may range from three square micron to 15 square micron, including from four square micron to 10 square micron, and from four square micron to eight square micron. Any serially deposited layer forming a portion of a construct described herein may be configured with any of these properties.

Further, the first layer 110 of the medical appliance 100 may comprise a thickness between 0.08 mm and 0.15 mm. By keeping the thickness of the first layer 110 of the medical appliance 100 between 0.08 mm and 0.15 mm, the loading attributes of the medical appliance 100 are improved. For example, the small thickness of the first layer 110 enables the medical appliance 100 to be crimped tighter and achieve a smaller French size when the medical appliance 100 is crimped and constrained for delivery in a delivery catheter.

In the illustrated embodiment, the medical appliance 100 comprises a second layer 120 disposed radially around the first layer 110. The second layer 120 may be configured to reinforce or otherwise strengthen the first layer 110 of rotational spun material. In some embodiments, this second layer 120 may comprise ePTFE. The ePTFE layer may reinforce the rotational spun first layer 110. For example, the ePTFE layer may increase the tensile strength, burst strength, hoop strength, or other properties of the medical appliance 100 as compared to a device utilizing only serially deposited fibers.

In some embodiments, the ePTFE may comprise densified ePTFE and/or ePTFE with a small IND. The ePTFE second layer 120 may be configured with low porosity and high work history to create a high-strength layer.

ePTFE layers within the scope of this disclosure may be configured with a variety of properties. For example, ePTFE layers with an average internodal distance (IND) of less than 80 micron, including less than 70 micron, less than 60 micron, less than 50 micron, less than 40 micron, less than 30 micron, less than 20 micron, and less than 10 micron, are within the scope of this disclosure. Additionally, exemplary ePTFE layers within the scope of this disclosure may have an average IND of less than 10, nine, eight, seven, six, five, four, three, two, or one micron.

Additionally, ePTFE layers within the scope of this disclosure may have percent porosities between 40% and 80%, including percent porosities greater than 50%. Further, such layers may have an average pore diameter greater than 1 micron, including between one micron and three micron. Average pore area may be greater than two square micron, including between two square micron and 15 square micron. Finally, the average fibril diameter of the ePTFE layer may be greater than 0.2 micron, including between 0.2 micron and 0.6 micron.

One exemplary ePTFE layer may have an IND of 10 micron, a percent porosity of 75%, average pore diameter of 4.38 micron, average pore area of 14.7 square micron, and average fibril thickness of 0.33 micron. A second exemplary ePTFE layer may have an IND of 10 micron, a percent porosity of 65%, average pore diameter of 3.5 micron, average pore area of 10.6 square micron, and average fibril thickness of 0.35 micron. A third exemplary ePTFE layer may have an IND of 10 micron, a percent porosity of 50%, average pore diameter of 2.78 micron, average pore area of 7.5 square micron, and average fibril thickness of 0.5 micron.

ePTFE layers according to the three exemplary layers above, as well as ePTFE layers within any of the ranges disclosed herein, may be used in multilayered constructs within the scope of this disclosure.

As compared to serially deposited layers, ePTFE layers may have a greater tensile strength, creep resistance, or other mechanical properties. For example, a multilayered construct may be comprised of both serially deposited layers and ePTFE layers wherein at least one ePTFE layer has five to 10 times greater tensile strength than at least one serially deposited layer within the same construct.

Multilayered coverings or constructs wherein one or more layers of ePTFE provide at least 90% of at least one mechanical property (such as tensile strength, hoop strength, burst strength, and/or creep resistance) of the covering or construct are within the scope of this disclosure. In some instances such a construct may have no scaffolding structure. In other embodiments, the comparison of properties may only refer to the properties provided by the covering portion, meaning the layers of material disposed about a scaffolding structure, but not including the scaffolding structure. Still further, this comparison of properties may refer to the properties of the entire construct, including a scaffolding structure. Furthermore, coverings or constructs wherein at least 85%, 80%, 75%, 70%, and 65% of at least one mechanical property is provided by one or more layers of ePTFE are within the scope of this disclosure.

In the illustrated embodiment, the scaffolding structure 130 is disposed around the second layer 120 of the medical appliance 100. This scaffolding structure 130 may comprise a metal stent-for example, a stent composed of nitinol, stainless steel, or alloys thereof. Additionally, other materials, such as polymer scaffolds, are within the scope of this disclosure. In some embodiments, the scaffolding structure 130 may be understood as a third layer of the medical appliance 100. For example, as the scaffolding structure 130 may be disposed between other such layers (such as the second layer 120 and the fourth layer 140) is can be understood as representing a layer of the medical appliance 100. Still further, in some embodiments the scaffolding structure may comprise a relative tight lattice, including a polymer lattice, which may tend to form a layer of a medical appliance 100. References herein to a multilayered construct or multilayered component may be understood as referring to the entire medical appliance 100, including the scaffolding structure, or may apply only to layers of material (such as 110, 120, 140, and 150) disposed about a scaffolding structure 130 considered separately from the scaffolding structure 130.

The medical appliance 100 may further comprise an impermeable layer, such as a tie layer. For example, in the illustrated embodiment, the fourth layer 140 may comprise a polymer layer that is impermeable to cellular growth, fluid passage, or both. An impermeable layer may be configured to prevent fluid leakage across the medical appliance, prevent cellular growth across the appliance, and/or prevent tissue growth into the impermeable layer. Containment of cellular growth across the medical appliance 100 may lengthen the useful life of the medical appliance 100, as bodily tissues are prevented from growing through the medical appliance 100 and occluding the lumen thereof.

In some embodiments, the impermeable fourth layer 140 may comprise fluorinated ethylene propylene (FEP) that may be sprayed, dipped, or laminated onto the construct. FEP layers within the scope of this disclosure may thus be applied as a film or membrane which is rolled onto or otherwise applied to a construct, as well as applied as a liquid or solution applied by spraying or dipping. In some embodiments, the impermeable fourth layer 140 may be a silicone, a urethane, a polyurethane, a fluoropolymer, an elastomer, or any other polymer that is biocompatible.

The medical appliance 100 may further comprise a fifth layer 150 disposed around the impermeable fourth layer 140. This fifth layer 150 may be an outermost layer of the medical appliance 100 and may define an abluminal surface of the medical appliance 100. In some instances, this fifth layer 150 may comprise ePTFE and may be densified and/or have a relatively small IND or pore size. This layer may be configured to provide strength to the construct and may or may not comprise ePTFE having the same properties as the second layer 120 of the illustrated construct.

In some embodiments, some of the layers may be helically wrapped with one or more strips of material. For example, the second layer 120 and the fifth layer 150 of the medical appliance 100 may be helically wrapped with one or more strips of ePTFE. FIG. 3D illustrates an exemplary strip 160 of ePTFE material. The strip 160 comprises a longitudinal direction (illustrated by arrow A1) and a transverse direction (illustrated by arrow A2). The strip 160 of ePTFE may be narrower than the length of the medical appliance 100. The strip 160 may be expanded or stretched in the longitudinal direction A1 of the strip 160 to create nodes 162 of material with fibrils 164 of material extending there between in the longitudinal direction A1 of the strip 160. For ease of explanation and illustration, the nodes 162 and the fibrils 164 are illustrated at a 950× magnification so the orientation of the nodes 162 and fibrils 164 are easily visible. However, the length of the strip 160 is not proportional to the size of the nodes 162 and fibrils 164.

The fibrils 164 of the ePTFE material creates tensile strength in the direction of the fibrils 164, i.e., the direction of expansion and stretching. In other words, after the ePTFE has been expanded in the longitudinal direction A1, the fibrils 164 are set in the longitudinal direction A1. The expansion or stretching of the ePTFE strain hardens the strip 160 and increases the tensile strength of the strip 160 and eliminates the elasticity of the strip 160, as discussed in more detail below.

The direction of the fibrils 164 oriented in the longidutianal direction A1 allows for the strip 160 to be expanded in the transverse direction A2. In other words, the transverse direction A2 of the strip 160 is not strain hardened, thus maintaining elasticity enabling the strip 160 to return to its shape in the transverse direction A2 when expanded in that direction but not beyond its yield strength. Accordingly, the tensile strength of the one or more strips 160 of ePTFE is greater in the longitudinal direction A1 of the one or strips 160 than in the transverse direction A2 of the one or more strips 160 of ePTFE.

In some embodiments, the strip 160 is only strain hardened in the longitudinal direction A1. In some embodiments, the strip 160 may be strain hardened in the longitudinal direction A1 creating fibrils 164 in the longitudinal direction A1 and also strain hardened in the transverse direction A2 creating fibrils 164 in the transverse direction A2. However, a majority of the fibrils 164 of the strip 160 are still oriented in the longitudinal direction A1 of the strip 160. For example, more than 50 percent of the fibrils 164 are oriented in the longitudinal direction A1 of the strip 160 than are oriented in the transverse direction A2 of the strip 160. In some embodiments, the ratio of strain hardening of the strips 160 in the longitudinal direction A1 versus the transverse direction A2 may be 10:1, 20:1, or 30:1. Accordingly, the ratio of the orientation of fibrils in the longitudinal direction A1 versus the transverse direction A2 may be 10:1, 20:1, or 30:1.

FIGS. 3E and 3F illustrate the fifth layer 150 of the medical appliance 100 with one or more strips 160 of ePTFE helically wrapped around the fourth layer 140 (not shown) of the medical appliance 100 forming the fifth layer 150 of the medical appliance 100. For comparison, the nodes 162 and the fibrils 164 of the one of more strips 160 are not illustrated in FIG. 3E but are illustrated in FIG. 3F. The second layer 120 of the medical appliance 100 may also comprise one or more strips 160 of ePTFE helically wrapped around the first layer 110 in a similar manner as the fifth layer 150 illustrated in FIGS. 3E and 3F.

The strips 160 are helically wrapped around the medical appliance 100 at a predetermined angle θ1. The angle θ1 may be 30° or less relative to a longitudinal axis 102 of the medical appliance 100. In other words, the strips 160 are helically wrapped around the medical appliance 100 at the predetermined angle θ1 between 0° and 30° relative to the longitudinal axis 102 of the medical appliance 100. Accordingly, the fibrils 164 of the strips 160 of ePTFE are aligned at an angle between 0° and 30° relative to the longitudinal axis 102 of the medical appliance 100. In some embodiments, the predetermined angle θ1 of the strips 160 helically wrapped around the medical appliance 100 is between 5° and 30° relative to the longitudinal axis 102 of the medical appliance 100. In some embodiments, the predetermined angle θ1 of the strips 160 helically wrapped around the medical appliance 100 is between 5° and 15° relative to the longitudinal axis 102 of the medical appliance 100. FIG. 3F illustrates that a majority of the fibrils 164 of the strips 160 are oriented at the predetermined angle θ1.

While the one or more strips 160 are illustrated as helically wrapped around the medical appliance 100 at the predetermined angle θ1 relative to the longitudinal axis 102 to the right, the one or more strips 160 may be helically wrapped around the medical appliance 100 at the predetermined angle θ1 relative to the longitudinal axis 102 to the left as well.

As discussed above, when the medical appliance 100 is radially expanded beyond a nominal diameter of the medical appliance 100 up to a use diameter, the second layer 120 and the fifth layer 150 provide additional strength to the medical appliance 100 in the longitudinal direction of the medical appliance 100, but the second layer 120 and the fifth layer 150 allow dilation or radial expansion beyond the nominal diameter of the medical appliance 100 in the axial direction due to the predetermined angle θ1 of the fibrils 164 relative to the longitudinal axis 102 of the medical appliance 100. The lack of fibrils 164 in the transverse direction A2 of the strip 160 of the second layer 120 and the fifth layer 150 allows the medical appliance 100 to overdilate. Further, as the medical appliance 100 dilates beyond the nominal diameter of the medical appliance 100, the tensile strength of the fibrils 164 relative to the longitudinal axis 102 of the medical appliance 100 reduce foreshortening of the medical appliance 100 as the medical appliance 100 expands beyond the nominal diameter of the medical appliance 100. For example, in some embodiments, when the medical appliance 100 dilates up to 40 percent beyond the nominal diameter of the medical appliance 100, the medical appliance 100 may foreshorten less than 10 percent of the length of the medical appliance 100. In some embodiments, when the medical appliance 100 dilates up to 40 percent beyond the nominal diameter of the medical appliance 100, the medical appliance 100 may foreshorten less than 5 percent of the length of the medical appliance 100.

FIG. 3G illustrates an exemplary stress strain curve of the one or more strips 160. As discussed above, the strips 160 are PTFE that are expanded or stretched in the longitudinal direction A1 to form ePTFE. Strain in is illustrated on the x axis and stress is illustrated on the y axis. As the strips 160 are stretched in the longitudinal direction A1, the strips 160 surpass the yield strength and become strain hardened in the longitudinal direction A1. The strips 160 are stretched in the longitudinal direction A1 up to the ultimate strength of the ePTFE but not surpassing the ultimate strength of the ePTFE to avoid necking and fracture of the ePTFE strip 160. Because the one or more strips 160 are strain hardened, the strips 160 are no longer elastic. In the strain hardened state, the strips 160 have significant tensile strength in the longitudinal direction A1 and can limit foreshortening in the longitudinal direction of the medical appliance 100 when the medical appliance 100 radially expands. However, the strips 160 are not strain hardened in the transverse direction A2, therefore, the strips 160 maintain elasticity in the transverse direction A2. Therefore, the medical appliance 100 can radially expand and contract due to the elasticity of the strips 160 in the transverse direction A2.

In some embodiments, the fourth layer 140 and the fifth layer 150 may be constructed as a composite layer. For instance, a fifth layer 150 comprising ePTFE may be sprayed or dipped with FEP such that the FEP coats the ePTFE and fills in the pores and openings in the ePTFE. A composite layer of ePTFE and FEP may thus be configured with the properties and functions of both the fourth layer 140 and fifth layer 150.

Any of the layers discussed above (110, 120, 140, 150) may be comprised of one or more sublayers. For example, if the first layer 110 is comprised of serially deposited fibers, the first layer 110 may, in turn, comprise multiple sublayers of serially deposited fibers or fiber mats. In an exemplary embodiment, the first layer 110 may thus consist of a first sublayer comprising serially deposited fibers and a second sublayer also comprising serially deposited fibers. The sublayers may be deposited at different times during manufacture and/or may be sintered separately, for example. The first layer 110 may also include other materials, disposed between the sublayers, for example to aid in coupling the sublayers to each other. In some embodiments, marker bands or other radiopaque films or objects may be disposed between sublayers. Any number of sublayers may be combined within a single layer.

In some embodiments, the wall thickness of a multilayered covering for the medical appliance 100 may be between 50 micron and 500 micron, including between 50 micron and 450 micron, between 50 micron and 400 micron, between 50 micron and 350 micron, between 50 micron and 300 micron, between 50 micron and 250 micron, between 50 micron and 200 micron, between 50 micron and 150 micron, and between 75 micron and 125 micron.

The wall thickness of any individual layer within a multilayered construct may be between five micron and 100 micron, including between five micron and 75 micron, between five micron and 60 micron, between 25 micron and 75 micron, between 10 micron and 30 micron, and between five micron and 15 micron. Any layer described herein may fall within any of these ranges, and the thicknesses of each layer of a multilayered construct may be configured such that the total wall thickness of the covering falls within the ranges described above.

Multilayered constructs that have more or fewer layers than the exemplary medical appliance 100 are likewise within the scope of this disclosure. For example, constructs having two, three, four, five, six, seven, or more layers are all within the scope of this disclosure. In some embodiments, a single layer may be used to provide the characteristics associated with two or more layers in the exemplary medical appliance 100. For example, ePTFE with a sufficiently small IND, densified ePTFE, or ePTFE with thick walls may be substantially impermeable to tissue ingrowth and/or fluid passage. Essentially, any material, layer, or microstructure that stops biological tissue from growing from one side of the layer to the other side when evaluated in a clinically relevant in vivo model. Therefore, in some embodiments, a single layer of low-porosity ePTFE may provide the characteristics associated with the second layer 120, the fourth layer 140, and the fifth layer 150 of the exemplary medical appliance 100. A medical appliance comprising a single luminal layer of serially deposited fibers and a single layer of ePTFE is within the scope of this disclosure.

Further, the order of the layers may be varied. For example, any of the layers described in connection with the exemplary medical appliance 100 may be disposed in any relative order, with the exception that a layer configured as a blood-contacting layer of the device (if present) will be disposed on the luminal surface of the construct.

Furthermore, medical appliances within the scope of this disclosure may be manufactured in a variety of ways. Each layer may be individually formed then disposed on the construct, or one or more layers may be formed on the construct in the first instance. The method of manufacture described below may also be applicable for medical appliances with more than or less than 5 distinct layers. For example, the medical appliance 100 may only have a single helically wrapped layer of ePTFE strips 160.

With reference to the medical appliance 100 of FIGS. 3A-3C, a method of manufacture may comprise serially depositing PTFE fibers on a mandrel or other collection surface and sintering the fibers. This layer of serially deposited fibers may form the first layer 110 of the medical appliance 100.

A second layer 120 may then be applied to the medical appliance 100. This second layer 120 may comprise ePTFE and may be densified. In some embodiments, the ePTFE may be obtained as a sheet which is applied around the first layer 110. Additionally, in some embodiments the second layer 120 may be applied around the first layer 110 before the first layer 110 is sintered. Sintering of the first layer 110 while the second layer 120 is disposed therearound may facilitate bonding between the first layer 110 and the second layer 120. Similarly, in some embodiments, the second layer 120 may be applied as unsintered ePTFE around the first layer 110, which in turn may comprise unsintered serially deposited PTFE fibers. Accordingly, the first layer 110 and second layer 120 may be simultaneously sintered, which may facilitate bonding between the layers. Any layer of PTFE, including serially deposited layers and layers of ePTFE, may be applied as an unsintered layer.

The second layer 120 may be applied by obtaining one or more strips 160 of ePTFE that is narrower than the length of the medical appliance 100. The one or more strips 160 may be helically wrapped around the first layer 110 of the medical appliance 100. As discussed above, the strip 160 may be wrapped at about 0 degrees to 30 degrees relative to the longitudinal axis 102 of the medical appliance 100.

A metal or polymer scaffolding structure 130 may then be applied around the second layer 120 of the medical appliance 100. The metal or polymer scaoffolding structure 130 may be fabricated by obtaining a tube of metallic or polymeric material at a manufacturing diameter that is smaller than a use diameter of the medical appliance 100. The metallic or polymeric material may be a polymer, nitinol, stainless steel, alloys thereof, or any other suitable material.

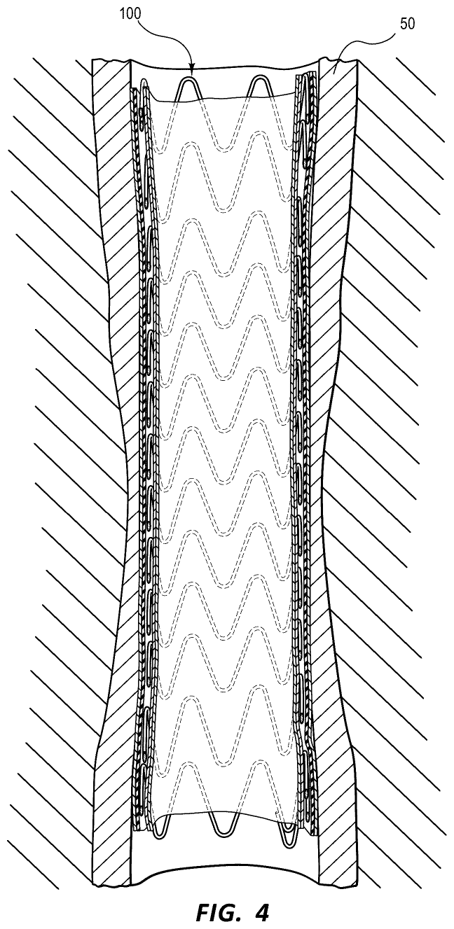

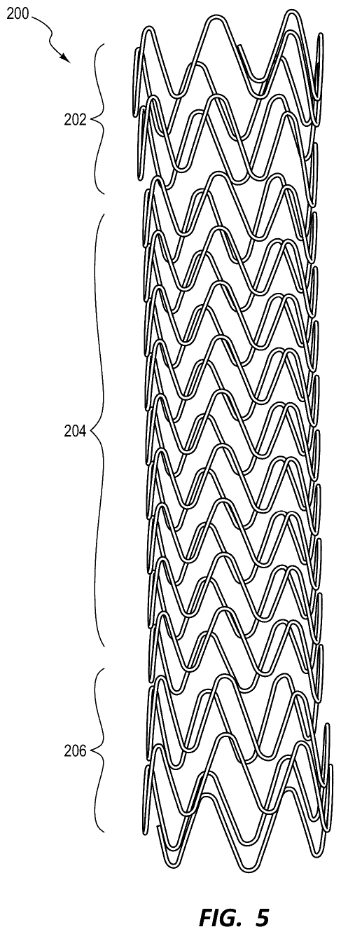

After obtaining the tube of metallic or polymeric material, the tube may be cut to form the stent frame or scaffolding structure 130 at the manufacturing diameter. The scaffolding structure 130 may be similar to the scaffolding structure 200 described below in relation to FIGS. 4 and 5. The scaffolding structure 130 may be formed using laser cutting, etching processes, and powdered metallurgy and sintering processes; formed from molds; and formed using rapid manufacturing techniques. The scaffolding structure 130 may have a variety of different shapes and forms, such as the shape disclosed in relation to scaffolding structure 200 described below in relation to FIGS. 4 and 5.

The fourth layer 140 may then be applied. The fourth layer 140 may comprise FEP and may be applied as a film, dipped, sprayed, or otherwise applied. Finally, the fifth layer 150 may be applied around the fourth layer 140. The fifth layer 150 may or may not be applied in the same manner as the second layer 120 and may or may not have substantially the same properties as the second layer 120.

In embodiments wherein the fifth layer 150 comprises PTFE, including ePTFE, the fifth layer 150 may or may not be sintered at the same time as other layers, for example, the second layer 120. In some embodiments, the first layer 110 and the second layer 120 may first be applied and sintered (either separately or simultaneously), the scaffolding structure 130 may then be applied, followed by the fourth layer 140. In some embodiments, the fourth layer 140 may comprise FEP and may be applied as a film or as a liquid or solution. In embodiments wherein the fourth layer 140 is FEP and the fifth layer 150 PTFE, such as ePTFE, the fifth layer 150 may be sintered after it is applied around the fourth layer 140. In some such embodiments, a film FEP fourth layer 140 may bond to adjacent layers during the heating of the construct to sinter the fifth layer 150. Again the first layer 110 and second layer 120 may be previously sintered. The fifth layer 150 may be applied by obtaining one or more strips 160 of ePTFE that is narrower than the length of the medical appliance 100. The one or more strips 160 may be helically wrapped around the fourth layer 140 of the medical appliance 100. As discussed above, the strip 160 may be wrapped at about 0 degrees to 30 degrees relative to the longitudinal axis 102 of the medical appliance 100.

After the medical appliance 100 is covered, the medical appliance 100 may be crimped down to a collapsed diameter. The collapsed diameter may be less than the diameter of the manufacturing diameter.

In some embodiments, after the tube is covered with the cover, the medical appliance 100 may be crimped down to a collapsed diameter on a first balloon. The collapsed diameter may be less than the diameter of the manufacturing diameter of the medical appliance 100. The first balloon is configured to expand the medical appliance 100. The first balloon may have a use diameter. The use diameter is the diameter of the first balloon when the first balloon is fully expanded. The use diameter of the first balloon may be the same as the manufacturing diameter of the medical appliance 100.

The medical appliance 100 may come in a kit for the medical practitioner to use with all of the components needed to successfully implant the medical appliance 100 to the target location of the patient. The kit may include the medical appliance 100 and a first balloon. In some embodiments, the medical appliance is crimped onto the first balloon. The medical appliance 100 comprises a nominal or manufacturing diameter and the first balloon comprises a nominal or use diameter. In some embodiments, the use diameter of the first balloon may be the same as the nominal diameter of the medical appliance 100. In some embodiments, the use diameter of the first balloon is greater than the nominal diameter of the medical appliance 100. The use diameter of the first balloon may be up to 40 percent beyond the nominal diameter of the medical appliance 100. In some embodiments, the use diameter of the first balloon may be between 20 percent and 40 percent greater than the nominal diameter of the medical appliance 100.

In some embodiments, the kit may further include a second balloon. The second balloon may have a use diameter or nominal diameter that is greater than the use diameter of the first balloon and is greater than the nominal diameter of the medical appliance 100. The use diameter of the second balloon may be up to 40 percent beyond the nominal diameter of the medical appliance 100. In some embodiments, the use diameter of the second balloon may be between 20 percent and 40 percent greater than the nominal diameter of the medical appliance 100.

The kit may further include additional medical devices, including snares, catheters, guidewires, introducers, needles, surgical equipment, and/or other items.

Methods of deploying a medical appliance, such as medical appliance 100, within the body are also within the scope of this disclosure. Similarly, methods of promoting endothelial growth while resisting transmural tissue growth across the medical appliance are within the scope of this disclosure. For example, deployment of a medical appliance having a blood-contacting layer configured to promote endothelial growth and at least one other layer configured to resist tissue growth through the layer would be related to such a method.

In one embodiment, the medical appliance 100 may be inserted to a target location in a patient's vasculature. The medical appliance 100 may be deployed and the medical appliance 100 may be expanded at the target location. In some embodiments, the medical appliance 100 may be a self-expanding stent or may be a balloon expandable stent. The medical appliance 100 may be expanded to a manufacturing diameter. In some situations, the medical practitioner may decide to expand the medical appliance 100 beyond the manufacturing diameter of the medical appliance 100 up to a use diameter. This may be for a variety of reasons. For example, the medical practitioner may expand the medical appliance 100 beyond the manufacturing diameter to apply further pressure to the vessel walls or plaque disposed within the vessel of the patient. Another reason is the greater range of dilation of the medical appliance 100 offers due to the ability of the medical appliance 100 to overdilate. This is extremely useful due to the changes in diameter of the patient's vasculature and the varying anatomies of different patients.

The use diameter of the medical appliance 100 is greater than the manufacturing diameter of the medical appliance 100. The use diameter of the medical appliance 100 may be up to 40 percent beyond the manufacturing diameter of the medical appliance 100. The medical appliance 100 maintains the imperviousness of the tie layer or fourth layer 140 up to use diameter, e.g., 40 percent beyond nominal. In other words, the medical appliance 100 passes the Water Entry Test based on ISO 7198 for testing vascular grafts, described above, at 40 percent beyond nominal. In some embodiments, the use diameter may be between 20 percent and 40 percent greater than the manufacturing diameter of the medical appliance 100.

In another embodiment, the medical appliance 100 may be inserted to a target location in a patient's vasculature. The medical appliance 100 may be deployed and the medical appliance 100 may be expanded at the target location. The medical appliance 100 may be expanded to the use diameter of the first balloon, which may be the same as the manufacturing diameter of the medical appliance 100. In some situations, the medical practitioner may decide to expand the medical appliance 100 to the manufacturing diameter of the medical appliance 100 with a second balloon. The second balloon may have a use diameter that is greater than the manufacturing diameter of the medical appliance 100. The medical practitioner may also expand the medical appliance 100 to a diameter beyond the manufacturing diameter of the medical appliance 100, up to a use diameter of the medical appliance 100. As discussed above, this may be done for a variety of different reasons. Accordingly, the medical practitioner may radially expand the medical appliance 100 from the use diameter of the first balloon, the manufacturing diameter of the medical appliance 100, or a use diameter of the second balloon.

The use diameter of the second balloon is greater than the manufacturing diameter of the medical appliance 100. The use diameter of the second balloon and the medical appliance 100 may be up to 40 percent beyond the manufacturing diameter of the medical appliance 100. In some embodiments, the use diameter of the second balloon and the medical appliance 100 may be between 20 percent and 40 percent greater than the manufacturing diameter of the medical appliance 100.

FIG. 4 is a cross-sectional schematic view of the medical appliance 100 deployed within a body lumen 50. As shown, when the medical appliance 100 is so deployed, the fifth layer (150 of FIG. 3A), which may comprise an abluminal surface of the medical appliance 100, may be disposed in direct contact with the wall of the body lumen 50. The first layer (110 of FIG. 3A), which may comprise a luminal surface of the medical appliance 100, may be disposed in direct communication with fluid flowing through the body lumen 50. Characteristics of the various other layers of the medical appliance 100 may also affect the interaction between the body lumen 50 and the medical appliance 100, though these layers may not be in direct contact with a surface of the body lumen 50. For example, a cellular or fluid impermeable layer may prevent tissue or fluid from crossing the wall of the medical appliance 100, though such a layer may or may not be disposed in direct contact with the body lumen 50.

FIG. 5 is a perspective view of a scaffolding structure 200 for a stent-graft. Such scaffolding structures may be coupled to coverings, including layered coverings, and may be configured to provide support and structure to the stent-graft. For example, a scaffolding structure, such as scaffolding structure 200, may be configured to resist radial compression of a stent-graft. Though references below may be directed to the scaffolding structure 200, it will be understood, by one having skill in the art and having the benefit of this disclosure, that disclosure relevant to the scaffolding structure 200 may analogously apply to a stent-graft or covered stent composed of the scaffolding structure 200.

In some embodiments, the scaffolding structure 200 may be configured with different resistance to radial force along the longitudinal length of the scaffolding structure 200. For example, in the illustrated embodiment, the scaffolding structure 200 comprises a proximal portion 202, a mid-body portion 204, and a distal portion 206. The scaffolding structure 200 may be configured such that it provides greater resistance to radial compression in one or more of these portions 202, 204, 206 as compared to at least one other portion 202, 204, 206 of the scaffolding structure 200. Differing resistance to radial force along the length of the scaffolding structure 200 may be designed to provide strength in certain areas (such as an area to be treated, such as an aneurysm) while providing softer portions of the scaffolding which may allow the scaffolding structure 200 to interact with healthy portions of the body in a more atraumatic way. Thus one or more portions of a scaffolding structure may be configured to hold open diseased tissue within the body lumen 50.

The radial resistance of the scaffolding structure 200, and any stent-graft comprising the scaffolding structure 200, may be a result of the material used to create the scaffolding structure 200; variations in the structure of the scaffolding structure 200, such as the degree to which the scaffolding structure 200 comprises a more open or more closed design; and other design parameters. In some embodiments within the scope of this disclosure, the radial force along portions of the same scaffolding structure, stent, or stent-graft may vary by between 10% and 30%, between 30% and 60%, between 60% and 100%, more than 100%, and more than 200%.

In some embodiments, the scaffolding structure 200 may be configured such that the resistance to radial compression of the scaffolding structure 200 is greater in the mid-body portion 204 of the scaffolding structure 200 as compared to the proximal portion 202 and/or distal portion 206 thereof. In other embodiments the mid-body portion 204 may have less resistance to radial compression or, in other words, may be softer than the proximal portion 202 and/or distal portion 206 thereof. Still further, in some embodiments the proximal portion 202 and/or distal portion 206 of a scaffolding structure 200 may be configured to reduce tissue aggravation at the edge of the scaffolding structure 200. In some instances the resistance to radial compression of one or more ends of the scaffolding structure 200 may be configured to reduce the occurrence of edge stenosis. Moreover, the resistance to radial compression of one or more ends of the scaffolding structure 200 may be configured to promote endothelial cell growth on a surface of a stent-graft coupled to the scaffolding structure 200. The resistance to radial compression along one or more portions of the scaffolding structure 200 may be configured to match the compliance of a body vessel in which the scaffolding structure 200 is designed for deployment.

Scaffolding structures 200 may comprise metals, including stainless steel, nitinol, various super elastic or shape memory alloys, and so forth. Scaffolding structures 200 may also comprise polymers. Further, scaffolding structures 200 may comprise one or more biologic agents, including embodiments wherein a metal or polymeric scaffolding structure is integrated with a drug or other biologic agent.

Scaffolding structures 200 may be formed in a variety of ways. In some embodiments, the scaffolding structure 200 may be formed of a wire. Further, the scaffolding structure 200 may be formed from a tube of material, including embodiments wherein the scaffolding structure 200 is cut from a tube of material. Scaffolding structures 200 may be formed using laser cutting, etching processes, and powdered metallurgy and sintering processes; formed from molds; and formed using rapid manufacturing techniques.

A stent or stent-graft, with or without a scaffolding structure such as scaffolding structure 200, may be configured to exert an outward radial force when disposed within a body lumen. This force may be configured to keep the lumen open, prevent restenosis, inhibit migration of the stent or stent-graft, and so forth. However, stents or stent-grafts which subject body lumens to high radial forces may provoke an unwanted biologic response and/or result in unnecessary trauma to the body lumen. Accordingly, a stent or stent-graft may be configured to exert radial force within a range that is acceptable for healing and trauma, while still achieving treatment goals.

Some stents or stent-grafts may be configured such that the stent or stent-graft resists localized compression, for example due to a point or pinch force, even when the localized force exceeds the circumferential outward radial force of the stent or stent-graft. In other words, the stent may be configured to resist relatively high point forces (for example, as may be exerted by a ligament or other biologic structure) on the stent or stent-graft without exerting high radial forces on the entire body lumen.

Stents or stent-grafts within the scope of this disclosure may be configured such that the point force required to fully collapse the stent is between five N and 15 N, including between 7.5 N and 12.5 N. Stents or stent-grafts within the point force ranges disclosed above may have a lower circumferential radial outward force. For example, stents or stent-grafts within the scope of this disclosure may have a radial outward force at 20% oversizing of between 0.3 N/mm and 1.3 N/mm, including between 0.4 N/mm and 1 N/mm. Thus, the point force required to fully collapse the stent may be significantly greater than the radial outward force exerted by the stent on a body lumen.

Multilayered stent covers within the scope of this disclosure may be tested by pressurizing water within the lumen of a stent according to the present disclosure. By increasing the water pressure, the burst strength and water permeability of the stent can be determined. Water entry pressure is herein defined as the internal water pressure at which a second bead of water forms on the outside of the tubular structure being tested. The device does not burst or fail in the water entry pressure test. Medical devices within the scope of this disclosure may have water entry pressures between zero psi and 10 psi, including between four psi and eight psi. Furthermore, medical devices within the scope of this disclosure may have water entry pressures greater than five psi, greater than 10 psi, greater than 20 psi, greater than 30 psi, greater than 40 psi, or greater than 50 psi.

In some embodiments, the scaffolding structure 200 may be formed from a tube of material, including embodiments wherein the scaffolding structure 200 is cut from a tube of material. Scaffolding structures 200 may be formed using laser cutting, etching processes, and powdered metallurgy and sintering processes; formed from molds; and formed using rapid manufacturing techniques.

The examples and embodiments disclosed herein are to be construed as merely illustrative and exemplary, and not as a limitation of the scope of the present disclosure in any way. It will be apparent to those having skill in the art with the aid of the present disclosure that changes may be made to the details of the above-described embodiments without departing from the underlying principles of the disclosure herein. It is intended that the scope of the invention be defined by the claims appended hereto and their equivalents.

1. A stent comprising:
a first layer of polytetrafluoroethylene (PTFE);
a second layer of expanded PTFE (ePTFE);
a third layer comprising a stent frame;
a fourth layer that is impervious to cell migration into the fourth layer;
a fifth layer of ePTFE;
wherein an order of the layers comprises the first layer as an innermost layer, the second layer, the third layer, the fourth layer, and the fifth layer as an outermost layer, and
wherein the second layer and the fifth layer each comprise one or more strips of ePTFE helically wrapped around the stent at an angle between 0 degrees and 30 degrees relative to a longitudinal axis of the stent.

2. The stent of claim 1, wherein the one or more strips of ePTFE are helically wrapped around the stent at an angle between 5 degrees and 30 degrees.

3. The stent of claim 1, wherein the stent is configured to dilate up to 40 percent beyond a nominal diameter of the stent.

4. The stent of claim 3, wherein when the stent dilates up to 40 percent beyond the nominal diameter of the stent, the stent is configured to foreshorten less than 10 percent of a length of the stent.

5. The stent of claim 1, wherein the one of more strips of ePTFE of the second layer and the fifth layer are only expanded in a longitudinal direction of the one or more strips.

6. The stent of claim 1, wherein the one or more strips of ePTFE of the second layer and the fifth layer are expanded in a longitudinal direction of the one or more strips and in a transverse direction of the one or more strips, and wherein a ratio of expansion of the one or more strips in the longitudinal direction versus the transverse direction is at least 10:1.

7. The stent of claim 1, wherein a majority of fibrils of the one or more strips of ePTFE of the second layer and the fifth layer are oriented at an angle between 0 degrees and 30 degrees relative to the longitudinal axis of the stent.

8. The stent of claim 1, wherein a tensile strength of the one or more strips of ePTFE is greater in a longitudinal direction of the one or strips than in a transverse direction of the one or more strips of ePTFE.

9. The stent of claim 1, wherein the first layer of PTFE has a thickness between 0.08 mm and 0.15 mm.

10. A covered stent comprising:
a stent frame; and
a cover coupled to the stent frame, comprising:
a first layer of polytetrafluoroethylene (PTFE);
a second layer of expanded PTFE (ePTFE); and
a tie layer,
wherein the second layer comprises one or more strips of ePTFE helically wrapped around the first layer, and
wherein a majority of fibrils of the one or more strips are oriented at an angle between 0 degrees and 30 degrees relative to a longitudinal axis of the covered stent.

11. The covered stent of claim 10, further comprising a third layer of ePTFE, wherein the third layer of ePTFE comprises one or more strips of ePTFE helically wrapped around the second layer, and wherein a majority of fibrils of the one or more strips of ePTFE of the third layer are oriented at an angle between 0 degrees and 30 degrees relative to the longitudinal axis of the covered stent.

12. The covered stent of claim 10, wherein the stent frame is disposed between the tie layer and the second layer.

13. The covered stent of claim 10, wherein the stent is configured to dilate up to 40 percent beyond a nominal diameter of the covered stent.

14. The covered stent of claim 13, wherein when the covered stent dilates up to 40 percent beyond the nominal diameter of the covered stent, the covered stent is configured to foreshorten less than 10 precent of a length of the covered stent.

15. The covered stent of claim 10, wherein the one of more strips of ePTFE of the second layer are only expanded in a longitudinal direction of the one or more strips of ePTFE.

16. The covered stent of claim 10, wherein the one or more strips of ePTFE of the second layer are expanded in a longitudinal direction of the one or more strips of ePTFE and in a transverse direction of the one or more strips of ePTFE, and
wherein a ratio of expansion in the longitudinal direction versus the transverse direction is at least 10:1.

17. The covered stent of claim 10, wherein the tie layer is disposed between the first layer and the second layer.

18. A method of manufacturing a covered stent comprising:
obtaining a tube of metallic material at a manufacturing diameter that is smaller than a use diameter;
cutting a stent frame from the tube of metallic material at the manufacturing diameter;
covering the stent frame with a cover at the manufacturing diameter to form a covered stent; and
crimping the covered stent to a collapsed diameter, which is less than the manufacturing diameter.

19. The method of claim 18, wherein the covered stent is configured to be balloon expandable to the use diameter up to 40 percent beyond the manufacturing diameter.

20. The method of claim 18, wherein the use diameter is 20 percent to 40 percent greater than the manufacturing diameter.
